(19) United States
(12) Patent Application Publication
(10) Pub. No.: US 2015/0150451 A1
(43) Pub. Date: Jun. 4, 2015

(54) DEVICE AND METHOD FOR VISUAL THRESHOLD MEASUREMENT

(71) Applicant: National Institute of Metrology, Beijing (CN)

(72) Inventors: Jiyan Zhang, Beijing (CN); Wenli Liu, Beijing (CN); Yan Chen, Beijing (CN); Zhenjie Li, Beijing (CN); Guiping Li, Beijing (CN); Jie Sun, Beijing (CN); Taogeng Zhou, Beijing (CN)

(21) Appl. No.: 14/407,584

(22) PCT Filed: Nov. 29, 2013
(86) PCT No.: PCT/CN2013/001469

§ 371 Date: Dec. 12, 2014

(30) Foreign Application Priority Data
Dec. 26, 2012 (CN) 201210571921.4

(51) Int. Cl. A61B 3/18; A61B 3/06; A61B 3/02; G01J 3/46

(52) U.S. Cl. CPC A61B 3/18; G01J 3/46; A61B 3/066; A61B 3/022; A61B 3/063; G02B 27/149

(57) ABSTRACT

The present invention relates to a device and method for visual threshold measurement, which belongs to the field of optical precision measurement technology. The device comprises a first uniform plane light source system, a second uniform plane light source system, a first primary color adjusting system, a second primary color adjusting system, a first complementary color adjusting system, a second complementary color adjusting system, a first color mixture system, a second color mixture system, and a beam splitting system. The device can generate four observation targets with variable brightness (illumination), contrast, color difference, and periodic pattern frequency, by using a four-integrating sphere light source system and by multiplexing the color adjusting systems, the color mixture systems, and subsequent light paths, so as to implement multi-parameter measurement for a set of visual thresholds. One device can simultaneously measure visual illumination, contrast, and color difference thresholds, and achieve ease operation, wide range measurement, multi-measurement-parameter coverage, large color gamut adjustment range, and high light source output power.

### FIELD OF THE INVENTION

The present invention relates to a device and method for visual threshold measurement, which belongs to the field of optical precision measurement technology.

### BACKGROUND OF THE INVENTION

More than 95% outside information consumed by human being are from the visual. Whether the visual characteristics are good is directly related to work and life. In clinical practices, the application of visual diagnostic equipments for early detection of visual disorders and early intervention treatments is very important. With the improvement of living standards, the emphasis on visual health is also increasing, and many new types of visual diagnostic equipments and testing technologies are developed, such as contrast sensitivity tester, chromatic meter and perimeter which are based on visual threshold measurement, and Electro-oculogram, Electroretinogram, Visual evoked potentiometer based on visual electrophysiological signals.

The existing visual diagnostic equipments such as contrast sensitivity tester, chromatic meter and perimeter based on visual threshold measurement, mostly perform single parameter test and their measuring range is limited. Different kinds of instruments need to be switched to implement illumination, contrast and color vision threshold examination, which is therefore complex, time-consuming and low efficient. Moreover, without unified calibration and traceability method, it is lack of comparability between measurement results given by different instruments, and mutual recognition of test results cannot be achieved among hospitals. Although relevant metrological research has been carried out for some visual diagnostic equipment and several national standards are established for optometry equipments such as refractometer, focimeter, refractor heads and so on, the calibration and traceability for visual diagnostic equipment based on visual threshold hasn't been solved yet. Further in-depth study on calibration and traceability method for visual diagnostic equipments is key to medical metrology during China's “twelfth five-year” period.

### SUMMARY OF THE INVENTION

The present invention is to provide a multi-functional integrated device for measurement of both vision illumination, contrast and color difference threshold of human eye, with output parameters realizing real-time control and accurate calibration. The invention of the device for measuring visual threshold can produce four observable targets: luminance (illumination), contrast, chromaticity and frequency variable cycle pattern, for accurate examination of illumination, contrast and color difference threshold of human eye.

The aim of the present invention is achieved by the following technical solutions.

The device used to measure visual threshold includes the first uniform plane light source, the second uniform plane light source, the first primary color adjusting system, the second primary color adjusting system, the first complementary color adjusting system, the second complementary color adjusting system, the first color mixture system, the second color mixture system and beam splitting system.

Among them, the first uniform plane light source includes the first light source and the first small integrating sphere; the second uniform plane light source includes the second light source and the second small integrating sphere; the first primary color adjusting system includes the first filter disc and the first block disc; the second primary color adjusting system is as same as the first one; the first complementary color adjusting system includes the second filter disc and the second block disc; the second complementary color adjusting system is as same as the first one; the first color mixture system includes the first big integrating sphere; the second color mixture system includes the second big integrating sphere; beam splitting system consists of a cubic splitting prism, target plate, the first reticle and the second reticle.

Each small integrating sphere has one input port and two exit ports which are at 90° to each other. Each big integrating sphere has one exit port, one detector port and two input ports which are at 90° to each other. Moreover, two exit ports of the first and second big integrating sphere are oriented at 90° to each other in the horizontal plane.

The connection relationship of the above parts is: The first light source is located at the input port of the first small integrating sphere and light enters into it through the input port. The first primary color adjusting system is located between the exit port of the first small integrating sphere and input port of the first big integrating sphere. The second primary color adjusting system is located between the other exit port of the first small integrating sphere and input port of the second big integrating sphere. Light with color and intensity of illumination based on three primary colors of red, green and blue is produced by joint action of color mixture system with the first and second primary color adjusting system. The second light source is located at the input port of the second small integrating sphere. The first complementary color adjusting system is located between the exit port of the second small integrating sphere and the other input port of the first big integrating sphere. The second complementary color adjusting system is located between the other exit port of the second small integrating sphere and the other input port of the second big integrating sphere. Another light with color and illumination is produced by joint action of color mixture system with the first and second complementary color adjusting system. However the three primary colors are not red, green and blue.

The exit port of the first big integrating sphere is directly facing one plane of the cubic splitting prism, and the exit port of the second big integrating sphere facing the other adjacent plane; the center of cubic splitting prism coincides with the intersection point of centerlines of exit port of the first and second big integrating spheres. The cubic splitting prism is used to generate observation targets by overlaying emergence light from the first and second big integrating sphere; Target plate is located close to the exit port of the first integrating sphere when taking contrast measurement. When taking color difference measurement, the first and second reticle, a set of conjugate reticle, is located close to two adjacent surfaces of cubic splitting prism with right angle. When observable for measurement of illumination and contrast threshold is generated, the target plate is used in the exit port of the first big integrating sphere, by switching different multiple target plates with different spatial frequency, observation targets with different spatial frequency can be generated.

Described target plates are with different spatial frequencies. By switching different target plate different observation targets can be generated.

The first and the second reticle described above have different patterns which are conjugate. They are used to generate two conjugate images with similar color for color difference measurement.

The described first primary color adjusting system consists of the first filter disc and the first block disc which are in turn placed parallel; the second primary color adjusting system has the same structure as the first one; the first complementary color adjusting system consists of the second filter disc and the second block disc which are placed in parallel too; the second complementary color adjusting system has the same structure as the first one.

The described first filter disc includes red filter, green filter and blue filter in order to generate red, green and blue primary colors. Each filter is fan shaped with 120° angle, and the plane constructed by the red, green and blue filter can completely block the exit port of first small integrating sphere and input port of the first big integrating sphere; The described second filter disc includes yellow filter, magenta filter and cyan filter in order to generate three other primary colors more than red, green and blue. Also the plane constructed by the yellow, magenta and cyan filter can completely block the exit port of second small integrating sphere and the other input port of the first big integrating sphere; The above six filters can be moved radially. The first block disc has the same structure as the first filter disc which includes three black light barriers, and the second block disc has the same structure as the second filter disc which also includes three black light barriers; the above six black light barriers can be moved radially too. By moving black light barriers radially of the first and second block discs the rays of light through the optical area will be changed, and then light with special color or illumination will be obtained at the exit port of big spheres.

In the described visual threshold measurement device, all interfaces should ensure no light leakage.

The method to realize the visual threshold measurement by the use of the device described in the present invention is.

(1) Color difference threshold measurement, the following provides specific steps:

The first light source light through the first small integrating sphere enters into the first primary color adjusting system. After the joint action of the first filter disc and block disc, light is into the first big integrating sphere. The second light source light through the second small integrating sphere enters into the first complementary color adjusting system. After the joint action of the second filter and block disc, the light is into the first big integrating sphere. Two beams of light are mixed by the first integrating sphere and a colored uniform light emits out of the exit port. The first light source light through the first small integrating sphere enters into the second primary color adjusting system. After the joint action of inside filter and block disc, the light is into the second big integrating sphere. The second light source light through the second small integrating sphere enters into the second complementary color adjusting system. After the joint action of inside filter and block disc, the light is into the second big integrating sphere. Two beams of light are mixed by the second integrating sphere and another uniform colored light emits out of the exit port. Then two beams of light pass through the first and second reticle respectively. After the action of cubic splitting prism observation target is generated for the measurement of color difference threshold.

Illumination threshold measurement, the following provides specific steps:

Method 1: The first light source light through the first small integrating sphere enters into the first primary color adjusting system. After the action of the color adjusting system, light is into the first big integrating sphere. Then light with special illumination emits out of its exit port. The uniform light shines on the target plate and observation target with different illumination for threshold measurement is obtained.

Method 2: The second light source light through the second small integrating sphere enters into the first complementary color adjusting system. After the action of the color adjusting system, light is into the first big integrating sphere. Light with special illumination emits out of its exit port. The uniform light shines on the target plate and observation target with different illumination for threshold measurement is obtained.

Method 3: The first light source light through the first small integrating sphere enters into the first primary color adjusting system and the first big integrating sphere in turn; the second light source light through the second small integrating sphere enters into the first complementary color adjusting system and the first big integrating sphere in turn; two beams of light is mixed by the first big integrating sphere and light with illumination emits out of the exit port. The uniform light shines on the target plate and observation target with different illumination for threshold measurement is obtained.

When taking illumination threshold measurement, filter discs in primary and complementary color adjusting systems should be all moved out and only black light barriers are in the optical path.

Contrast threshold measurement, the following provides specific steps:

Method 1: The first light source light through the first small integrating sphere enters into the first primary color adjusting system and the first big integrating sphere in turn. Light with special illumination emits out and shines on the target plate with different spatial frequency, and then enters into the cubic splitting prism. Observation targets with different spatial frequency can be obtained by switching different target plates. The second light source light through the second small integrating sphere enters into the second complementary color adjusting system and the second big integrating sphere in turn. Another light with special illumination emits out and enters into the cubic splitting prism. Two beams of light mix in the splitting system and observation target for contrast threshold measurement is obtained.

Method 2: The first light source light through the first small integrating sphere enters into the first primary color adjusting system and the first big integrating sphere in turn. Light with special illumination emits out and shines on the target plate with different spatial frequency, and then enters into the cubic splitting prism. Observation targets with different spatial frequency can be obtained by switching different target plates. The first light source light through the first small integrating sphere enters into the second primary color adjusting system and the second big integrating sphere in turn. Another light with illumination emits out and enters into the cubic splitting prism. Two beams of light mix in the splitting system and observation target for contrast threshold measurement is obtained.

Method 3: The second light source light through the second small integrating sphere enters into the first complementary color adjusting system and the first big integrating sphere in turn. Light with special illumination emits out and shines on the target plate with different spatial frequency, and then enters into the cubic splitting prism. Observation targets with different spatial frequency can be obtained by switching different target plates. The second light source light through the second small integrating sphere enters into the second complementary color adjusting system and the second big integrating sphere in turn. Another light with special illumination emits out and enters into the cubic splitting prism. Two beams of light mix in the splitting system and observation target is obtained for contrast threshold measurement.

Method 4: The second light source light through the second small integrating sphere enters into the first complementary color adjusting system and the first big integrating sphere in turn. Light with special illumination emits out and shines on the target plate with different spatial frequency, and then enters into the cubic splitting prism. Observation targets with different spatial frequency can be obtained by switching different target plates. The first light source light through the first small integrating sphere enters into the second primary color adjusting system and the second big integrating sphere in turn. Another light with illumination emits out and enters into the cubic splitting prism. Two beams of light mix in the splitting system and observation target is obtained for contrast threshold measurement.

When the contrast threshold measurement is performed, filter discs in primary and complementary color adjusting systems should be all moved out and only black light barriers are in the optical path.

Beneficial Effects

1. The present invention relates to a device and method for visual threshold measurement. By the use of the four integrating sphere system, color adjusting system, color mixture system and subsequent optical path, four observable targets including luminance (illumination), contrast, chromaticity and frequency variable cycle pattern can be generated. The device realizes measurement of illumination, contrast and color difference threshold simultaneously with the advantage of convenient operation, large color adjustment range and big light output power.

2. The present invention relates to a device and method for visual threshold measurement. Output parameters of the device can be real-time controlled and accurately calibrated by the use of metrological instruments such as spectrometer, illuminometer and so on. As a result, the calibration and traceability of the whole device can be realized and measurement results can be guaranteed to be accurate and reliable. On the basis of the achievement, standard device for visual threshold measurement can be established and calibration or traceability of visual threshold diagnostic equipments can be realized.

### BRIEF DESCRIPTION OF THE DRAWINGS

FIG. 1 is a left view of the spatial structure of the visual threshold measurement device in present invention;

FIG. 2 is a top view of the spatial structure of the visual threshold measurement device in present invention;

FIG. 3 is a schematic diagram for the visual threshold measurement device in present invention;

FIG. 4 is an inside structure of primary and complementary color adjusting system; where, “a” is for primary color adjusting system and “b” is for complementary color adjusting system;

FIG. 5 is an example for one application of the visual threshold measurement device in present invention;

FIGS. 6A-6C is an example for optical path design to generate observation targets of color difference measurement; FIG. 6A is pattern of the first reticle, FIG. 6B is pattern of the second reticle, and FIG. 6C is the schematic optical system;

FIG. 7 is an example for the design of target plates used for contrast threshold measurement.

Where, 1—the first light source, 2—the first small integrating sphere, 3—the second light source, 4—the second small integrating sphere, 5—the first big integrating sphere, 6—the second big integrating sphere, 7—the first primary color adjusting system, 8—the second primary color adjusting system, 9—the first complementary color adjusting system, 10—the second complementary color adjusting system, 11—cubic splitting prism, 12—target plate, 13—the first reticle, 14—the second reticle, 15—the first filter disc, 16—the first block disc, 17—the second filter disc, 18—the second block disc, 19—red filter, 20—greeen filter, 21—blue filter, 22—black light barrier, 23—yellow filter, 24—cyan filter, 25—magenta filter, 26—illuminometer, 27-spectrometer, 28—the first probe, 29—the second probe, 30—optical fiber.

### DETAILED DESCRIPTION OF THE EMBODIMENTS

The invention is further explained with the combination of accompanying figures and example.

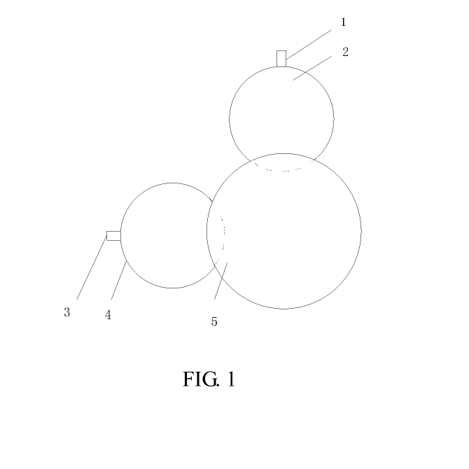

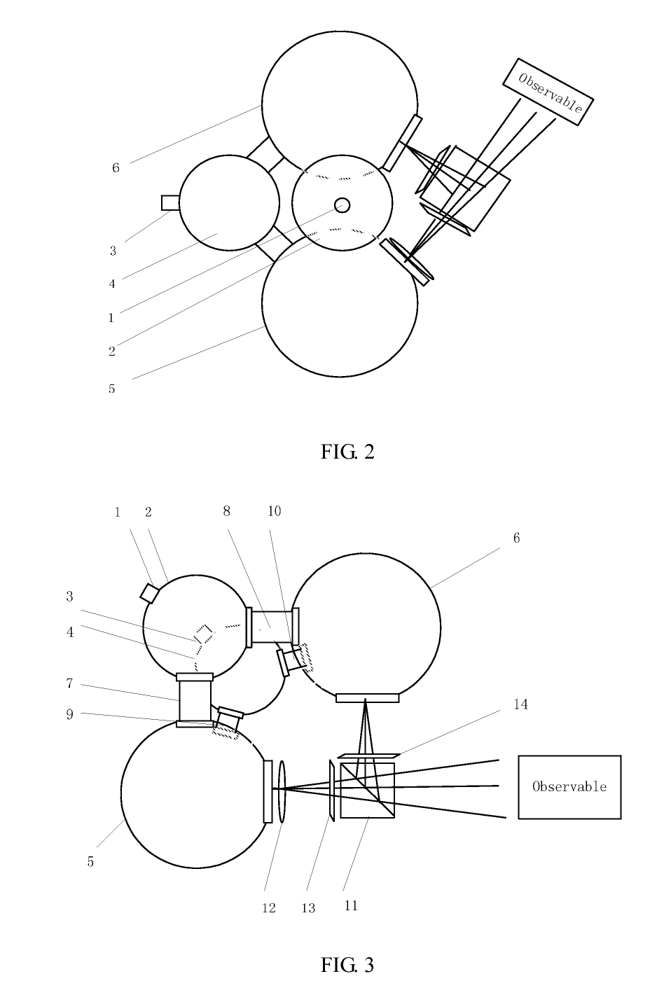

The device for visual threshold measurement consists of the first uniform plane light source (including the first light source 1 and the first small integrating sphere 2), the second uniform plane light source (including the second light source 3 and the second small integrating sphere 4), the first primary color adjusting system 7, the second primary color adjusting system 8, the first complementary color adjusting system 9, the second complementary color adjusting system 10, the first color mixture system (including the first big integrating sphere 5), the second color mixture system (including the second big integrating sphere 6) and beam splitting system (including cubic splitting prism 11, target plate 12, the first reticle 13 and the second reticle 14), as shown in FIG. 1, FIG. 2 and FIG. 3.

Each small integrating sphere has one input port and two exit ports which are at 90° to each other. Each big integrating sphere has one exit port, one detector port and two input ports which are at 90° to each other too. Moreover, two exit ports of the first and second big integrating spheres are oriented at 90° to each other in the horizontal plane.

The first light source 1 is located at the input port of the first small integrating sphere 2 and light enters into it through the input port. The first primary color adjusting system 7 is located between the exit port of the first small integrating sphere 2 and input port of the first big integrating sphere 5. The second primary color adjusting system 8 is located between the other exit port of the first small integrating sphere 2 and input port of the second big integrating sphere 6. Light with color and intensity of illumination based on the three primary colors of red, green and blue is produced by the joint action of color mixture system with the first and second primary color adjusting system. The second light source 3 is connected with the input port of the second small integrating sphere 4. The first complementary color adjusting system 9 is located between the exit port of the second small integrating sphere 4 and the other input port of the first big integrating sphere 5. The second complementary color adjusting system 10 is located between the other exit port of the second small integrating sphere 4 and the other input port of the second big integrating sphere 6. Light with color and illumination is produced by the joint action of color mixture system with the first and second complementary color adjusting system. However the three primary colors are not red, green and blue. The exit port of the first big integrating sphere 5 is directly facing one plane of the cubic splitting prism 11, and the exit port of the second big integrating sphere 6 facing the other adjacent right angle plane. The center of the cubic splitting prism 11 is coinciding with the intersection point of centerlines of exit port of the first 5 and second big integrating spheres 6.

The probe of illuminometer 26 is located at the exit port of the first big integrating sphere 5 in order to detect the exit surface illumination at real time. Dual-channel spectrometer 27 has two probes: the first probe 28 and the second probe 29. The first probe 28 is located at the detector port of the first big integrating sphere 5 and the second probe 29 is located at the detector port of the second big integrating sphere 6. Two probes are connected with spectrometer 27 with optical fiber 30, so that the spectral distribution of the light emitted from the first big integrating sphere 5 and the second big integrating sphere 6 can be real-time monitored, as shown in FIG. 5.

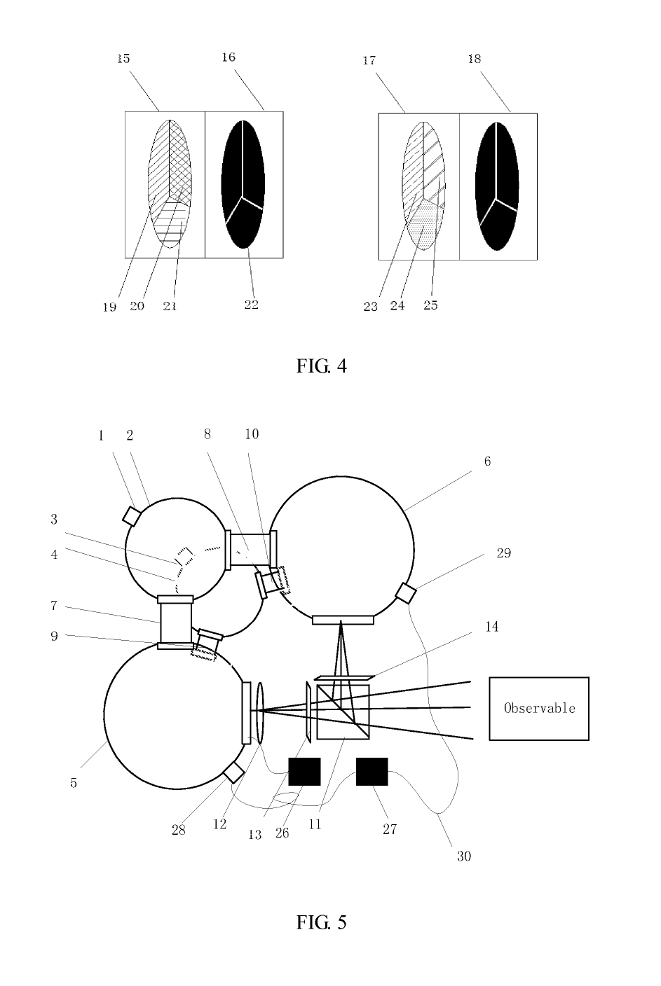

The first primary color adjusting system 7 consists of the first filter disc 15 and the first block disc 16 which are in turn placed parallel; the second primary color adjusting system has the same structure as the first one and inside structure is shown in FIG. 4A; the first complementary color adjusting system 9 consists of the second filter disc 17 and the second block disc 18 which are in turn placed parallel too; the second complementary color adjusting system has the same structure as the first one and inside structure is shown in FIG. 4B.

The first filter disc 15 includes red filter 19, green filter 20 and blue filter 21 in order to generate red, green and blue primary colors. Each filter is fan shaped with 120° angle, and the plane constructed by the red 19, green 20 and blue filter 21 can completely block the exit port of first small integrating sphere 2 and input port of the first big integrating sphere 5; The second filter disc 17 includes yellow filter 23, cyan filter 24 and magenta filter 25 in order to generate three other primary colors more than red, green and blue. Also the plane constructed by the yellow 23, cyan 24 and magenta filter 25 can completely block the exit port of the second small integrating sphere 4 and the other input port of the first big integrating sphere 5; The above six filters can be moved radially. The first block disc 16 has the same structure as the first filter disc 15 which includes three black light barriers 22, and the second block disc 18 has the same structure as the second filter disc 17 which also includes three black light barriers; the above six black light barriers can be moved radially too. By radially moving black light barriers of the first 16 and second block discs 18, the light through the optical areas can be changed, and then light with special color or illumination can be obtained at the exit port of big spheres.

In the example of the invention, the first light source 1 and the second light source 3 both use halogen lamp.

As shown in FIG. 5, the steps to generate four observables used for measurement of visual threshold are listed below:

First, open the power supply of the first 1 and second light source 3. Light and preheat the halogen lamps to illuminate stably. The first light source 1 and the first small integrating sphere 2 form the first uniform plane light source, and the second light source 3 and the second small integrating sphere 4 form the second uniform plane light source, which are used to produce uniform emergent light with continuous spectrum.

(1) The steps for color difference threshold measurement:

The first light source 1 light through the first small integrating sphere 2 enters into the first primary color adjusting system 7. After the joint action of the first filter disc 15 and block disc 16, the light is into the first big integrating sphere 5. The second light source 3 light through the second small integrating sphere 4 enters into the first complementary color adjusting system 9. After the joint action of the second filter disc 17 and block disc 18, the light is into the first big integrating sphere 5. Two beams of light are mixed by the first integrating sphere 5 and uniform colored light emits out of the exit port. The first light source 1 light through the first small integrating sphere 2 enters into the second primary color adjusting system 8. After the joint action of inside filter and block disc, the light is into the second big integrating sphere 6. The second light source 3 light through the second small integrating sphere 4 enters into the second complementary color adjusting system 10. After the joint action of inside filter and block disc, the light is into the second big integrating sphere 6. Two beams of light are mixed by the second integrating sphere 6 and another uniform colored light emits out of the exit port. Light emitted from the first big integrating sphere 5, through a frosted glass, shines on the first reticle 13 and a colored pattern is produced. Light emitted from the second big integrating sphere 6, through another frosted glass, shines on the second reticle 14 and another colored pattern is produced. Two beams of light pass through the cubic splitting prism 11 and mix. Observation target for color difference threshold measurement is then generated. A pattern with mixing color can be seen or detected by eyes or equipment under test.

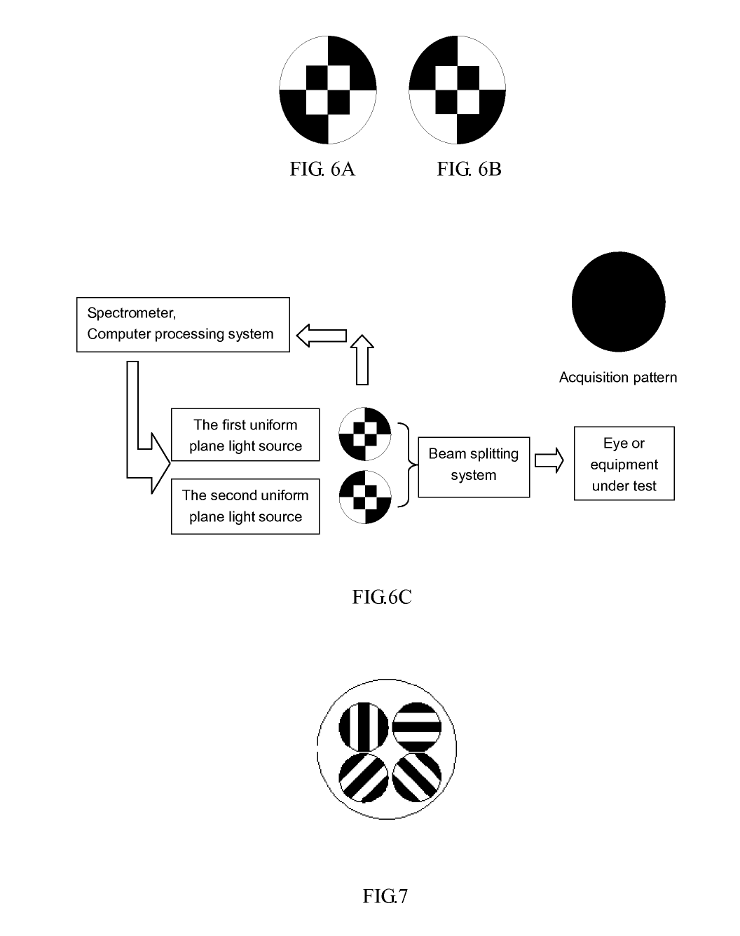

By the use of fiber optic spectrometer, the specific color parameters of the two patterns can be measured, such as Lab chromaticity coordinate, X/Y/Z value and so on. Then the color difference ΔE for these two colors is calculated with computer. With the control of the color of emitted light from the first and second uniform plane light source, the color difference ΔE can be changed close to the threshold of eye or equipment under test, as shown in FIG. 6C.

The conjugate pattern on two sets of reticles is shown in FIGS. 6A and 6B, 6A is the pattern on the first reticle and 6B is that on the second reticle. Wherein the white part is the transparent glass substrate and black part is the opaque coating.

With the first probe 28 and the second probe 29 of dual-channel spectrometer connected, the color of emitted light from the first 5 and second big integrating sphere 6 is real-time monitored. With the feedback to the first primary color adjusting system 7, the second primary color adjusting system 8, the first complementary color adjusting system 9 and the second complementary color adjusting system 10, light through the optical area is changed by radially moving the inside black light barriers and corresponding color light is obtained.

(2) The steps for illumination threshold measurement:

The first light source 1 light through the first small integrating sphere 2 enters into the first primary color adjusting system 7. With the action of block disc 16, light with special illumination is emitted out of the exit port of the first big integrating sphere 5 and observation target with different illumination for threshold measurement is obtained.

Light emitted from the first big integrating sphere 5 is real-time monitored by illuminometer 26. With the feedback of the light illumination to the first primary color adjusting system 7, light through the optical area is changed by radially moving the inside black light barriers and as a result corresponding illumination is obtained.

(3) The steps for contrast threshold measurement:

The first light source 1 light through the first small integrating sphere 2 enters into the first primary color adjusting system 7. With the action of the first block disc 15, light with special illumination emitted from the first big integrating sphere 5 shines on the target plate 12 with different spatial frequency, and then enters into the cubic splitting prism 11. The second light source 3 light through the second small integrating sphere 4 enters into the second complementary color adjusting system 10. With the action of the inside block disc, another light with special illumination emits out of the second big integrating sphere 6 and enters into the cubic splitting prism 11. Two beams of light mix in the splitting system and observation target is obtained for contrast threshold measurement. In the example of this invention, a kind of transmissive grating optotype is designed for contrast threshold measurement according to the Weber-Fechner law, which includes five different spatial frequency listed in table 1. They are scored on five target plates and there are four different direction (0°, 45°, 90° and 135°) of grating strips for each spatial frequency target. An example of the target plate is shown in FIG. 7.

TABLE 1
Spatial frequency and cycle

| No. | spatial frequency c/d | cycle (mm) |
|---|---|---|
| 1 | 1.5 | 3.49 |
| 2 | 3.0 | 1.75 |
| 3 | 6.0 | 0.87 |
| 4 | 12.0 | 0.44 |
| 5 | 18.0 | 0.36 |

When observable for measurement of illumination and contrast threshold is generated, only black light barriers in the primary and complementary color adjusting systems are used and inside filters are all moved out of the optical path completely.

The present invention relates to a device and method for visual threshold measurement by generating four observable targets including luminance (illumination), contrast, chromaticity and frequency variable cycle pattern. By comparison with other test methods it has the advantage of convenient operation, simple structure, large color adjustment range and big light output power. Moreover, output parameters of the device can be real-time controlled and accurate calibrated, which is very important for realization of calibration and traceability for quantity values. In this way measurement results are guaranteed to be accurate and reliable.

Above in connection with the accompanying figures, an example of the invention has been explained; however these instructions should not be construed as scope limitation of the invention. The protection scope of the invention is defined and limited by the appended claims, and any modifications based on the claims are within the protection scope of the invention.

1. An apparatus for measuring visual threshold, comprising:
a first uniform plane light source system comprising a first small integrating sphere having an input port for receiving light source, a first exit and a second exit ports;
a second uniform plane light source system comprising a second small integrating sphere having an input port for receiving light source, a first exit and a second exit ports;
a first color mixture system comprising a first big integrating sphere having a first input port, a second input port and an output port;
a second color mixture system comprising a second big integrating sphere having a first input port, a second input port and an output port;
a first primary color adjusting system comprising a light filtering disc and a light blocking disc and disposed between the first exit port of the first small integrating sphere and the first input port of the first big integrating sphere;
a second primary color adjusting system comprising a light filtering disc and a light blocking disc and disposed between the second exit port of the first small integrating sphere and the first input port of the second big integrating sphere;
a first complementary color adjusting system comprising a light filtering disc and a light blocking disc and disposed between the first exit port of the second small integrating sphere and the second input port of the first big integrating sphere;
a second complementary color adjusting system comprising a light filtering disc and a light blocking disc and disposed between the second exit port of the second small integrating sphere and the second input port of the second big integrating sphere; and
a beam splitting system comprising of a cubic splitting prism having a first and a second adjacent planes, a target plate disposed in a proximity to the exit port of the first big integrating sphere for taking contrast and illumination measurement, a first reticle and a second reticle disposed in a proximity to the respective first and second adjacent planes of the cubic splitting prism for taking color difference measurement;
wherein the exit port of the first big integrating sphere is configured to face the first adjacent plane of the cubic splitting prism, and the exit port of the second big integrating sphere is configured to face the second adjacent plane of the cubic splitting prism such that the center of the cubic splitting prism coincides with the intersection point of centerlines of the exit port of the first big integrating sphere and the second big integrating spheres.

2. The apparatus of claim 1, wherein:
the axes of the first and second exit ports of the first small integrating sphere are perpendicular to each other;
the axes of the first and second exit ports of the second small integrating sphere are perpendicular to each other;
the first and second big integrating spheres each having a detector port; and
the axes of the exit port of the first big integrating sphere and the exit port of the second big integrating sphere are perpendicular to each other.

3. The apparatus of claim 1, wherein the target plate is adapted to vary by spatial frequency to generate observation targets with different spatial frequency.

4. The apparatus of claim 1, wherein the first reticle and the second reticle are adapted to have conjugate patterns for generating two conjugate images with similar color.

5. The apparatus of claim 1, wherein the light filtering disc and the light blocking disc in the first primary color adjusting system and the second primary color adjusting system are each disposed in parallel to each other, respectively, the light filtering disc and the light blocking disc in the first complementary color adjusting system and the second complementary color adjusting system are each disposed in parallel to each other, respectively.

6. The apparatus of claim 1, wherein the light filtering disc in the first primary color adjusting system and the second primary color adjusting system is adapted to include a red filter, a green filter and a blue filter, each of the filters being a fan shape with 120° angle and movable radially to form a plane that completely covers light going through each corresponding primary color adjusting system;
the light filtering disc in the first complementary color adjusting system and the second complementary color adjusting system is adapted to include a yellow filter, a magenta filter and a cyan filter, each of the filters being a fan shape with 120° angle to form a plane that completely covers light going through each corresponding complementary color adjusting system;
the light blocking discs in the first and second primary color adjusting systems and the first and second complementary color adjusting systems are configured to each include three black light barriers, each being a fan shape with 120° angle and movable radially to change light going through each corresponding color adjusting system therefore to obtain light with special color or illumination at the exit port of the first or second big integrating sphere connected thereto.

7. A method for measuring at lease one of a plurality of visual thresholds comprising a color difference threshold, an illumination threshold and a contrast threshold, by disposing an apparatus comprising:
a first uniform plane light source system comprising a first small integrating sphere having an input port for receiving light source, a first exit and a second exit ports;
a second uniform plane light source system comprising a second small integrating sphere having an input port for receiving light source, a first exit and a second exit ports;
a first color mixture system comprising a first big integrating sphere having a first input port, a second input port and an output port;
a second color mixture system comprising a second big integrating sphere having a first input port, a second input port and an output port;
a first primary color adjusting system comprising a light filtering disc and a light blocking disc and disposed between the first exit port of the first small integrating sphere and the first input port of the first big integrating sphere;
a second primary color adjusting system comprising a light filtering disc and a light blocking disc and disposed between the second exit port of the first small integrating sphere and the first input port of the second big integrating sphere;
a first complementary color adjusting system comprising a light filtering disc and a light blocking disc and disposed between the first exit port of the second small integrating sphere and the second input port of the first big integrating sphere;
a second complementary color adjusting system comprising a light filtering disc and a light blocking disc and disposed between the second exit port of the second small integrating sphere and the second input port of the second big integrating sphere;
a beam splitting system comprising of a cubic splitting prism having a first and a second adjacent planes, a target plate disposed in a proximity to the exit port of the first big integrating sphere, a first reticle and a second reticle disposed in a proximity to the respective first and second planes of the cubic splitting prism, wherein the exit port of the first big integrating sphere directly faces the first adjacent plane of the cubic splitting prism, and the exit port of the second big integrating sphere faces the second adjacent plane of the cubic splitting prism such that the center of the cubic splitting prism coincides with the intersection point of centerlines of the exit port of the first big integrating sphere and the second big integrating spheres;
whereby the measuring of the color difference threshold comprising the steps of:
(1) receiving a first light source at the first uniform plane light source system and allowing the first light source to travel through the first primary color adjusting system to the first color mixture system, receiving a second light source at the second uniform plane light source system and allowing the second light source to travel through the first complementary color adjusting system to the first color mixture system, mixing the beams of the first light source and the second light source in the first color mixture system to emit a first colored uniform light;
(2) receiving the first light source at the first uniform plane light source system and allowing the first light source to travel through the second primary color adjusting system to the second color mixture system, receiving the second light source at the second uniform plane light source system and allowing the second light source to travel through the second complementary color adjusting system to the second color mixture system, mixing the beams of the first light source and the second light source in the second color mixture system to emit a second colored uniform light; and
(3) passing the first colored uniform light through the first reticle to the cubic splitting prism, passing the second colored uniform light through the second reticle to the cubic splitting prism and mixing the first and second colored uniform light at the cubic splitting prism to generate an observation target for measuring the color difference threshold;
the measuring of the illumination threshold comprising one of the steps of:
(1) receiving a light source at the first uniform plane light source system and allowing the light source to travel through the first primary color adjusting system to the first color mixture system and emitting a uniform light on the target plate to form an observation target with illumination for measuring the illumination threshold;
(2) receiving a light source at the second uniform plane light source system and allowing the light source to travel through the first complementary color adjusting system to the first color mixture system and emitting a uniform light on the target plate to form an observation target with illumination for measuring the illumination threshold; and
(3) receiving a first light source at the first uniform plane light source system and allowing the first light source to travel through the first primary color adjusting system to the first color mixture system, receiving a second light source at the second uniform plane light source system and allowing the second light source to travel through the first complementary color adjusting system to the first color mixture system, mixing the first light source and the second light source in the first color mixture system, and emitting a uniform light on the target plate to form an observation target with illumination for measuring the illumination threshold; and
the measuring of the contrast threshold comprising one of the steps of:
(1) receiving a first light source at the first uniform plane light source system and allowing the first light source to travel through the first primary color adjusting system to the first color mixture system to emit a first uniform light to go through the target plate to the cubic splitting prism, receiving a second light source at the second uniform plane light source system and allowing the second light source to travel through the second complementary color adjusting system to the second color mixture system to emit a second uniform light to go through the cubic splitting prism, and mixing the first and second emitted uniform light to form an observation target for measuring the contrast threshold measurement;
(2) receiving a light source at the first uniform plane light source system and allowing the light source to travel through the first primary color adjusting system to the first color mixture system to emit a first uniform light to go through the target plate to the cubic splitting prism, allowing the light source at the first uniform plane light source system to travel through the second primary color adjusting system to the second color mixture system to emit a second uniform light to go through the cubic splitting prism, and mixing the first and second emitted uniform light to form an observation target for measuring the contrast threshold measurement;
(3) receiving a light source at the second uniform plane light source system and allowing the light source to travel through the first complementary color adjusting system to the first color mixture system to emit a first uniform light to go through the target plate to the cubic splitting prism, allowing the light source at the second uniform plane light source system to travel through the second complementary color adjusting system to the second color mixture system to emit a second uniform light to go through the cubic splitting prism, and mixing the first and second emitted uniform light to form an observation target for measuring the contrast threshold measurement; and
(4) receiving a second light source at the second uniform plane light source system and allowing the second light source to travel through the first complementary color adjusting system to the first color mixture system to emit a first uniform light to go through the target plate to the cubic splitting prism, receiving a first light source at the first uniform plane light source system and allowing the first light source to travel through the second primary color adjusting system to the second color mixture system to emit a second uniform light to go through the cubic splitting prism, and mixing the first and second emitted uniform light to form an observation target for measuring the contrast threshold measurement;
wherein the light filtering disc in the first and second primary color adjusting systems and the first and second complementary color adjusting systems is removed from the optical path when measuring the illumination threshold or the contrast threshold.

8. The apparatus of claim 1, wherein:
the first big integrating sphere comprising a probe for real-time monitoring of color of the light therein, wherein the black light barriers of the first primary color adjusting system and the black light barriers of the first complementary color adjusting system are adapted to adjust the light going through based on the monitored color inside the first big integrating sphere; and
the second big integrating sphere comprising a probe for real-time monitoring of color of the light therein, wherein the black light barriers of the second primary color adjusting system and the black light barriers of the second complementary color adjusting system are adapted to adjust the light going through based on the monitored color inside the second big integrating sphere.

9. The method of claim 7, wherein the measuring of the contrast threshold further comprising using the target plate selected from a group of target plates comprising a plurality of Weber-Fechner transmissive grating optotypes corresponding to different spatial frequencies, each grating optotype further comprising one or more grating strip patterns.

10. The method of claim 9, wherein the group of target plates comprising five Weber-Fechner transmissive grating optotypes and each grating optotype further comprising four grating strip patterns corresponding to 0°, 45°, 90° and 135°, respectively.

11. An apparatus for measuring a plurality of visual thresholds, comprising:
a first uniform plane light source system comprising a first small integrating sphere having an input port for receiving light source, a first exit and a second exit ports;
a second uniform plane light source system comprising a second small integrating sphere having an input port for receiving light source, a first exit and a second exit ports;
a first color mixture system comprising a first big integrating sphere having a first input port, a second input port and an output port;
a second color mixture system comprising a second big integrating sphere having a first input port, a second input port and an output port;
a first primary color adjusting system comprising a light filtering disc and a light blocking disc and disposed between the first exit port of the first small integrating sphere and the first input port of the first big integrating sphere;
a second primary color adjusting system comprising a light filtering disc and a light blocking disc and disposed between the second exit port of the first small integrating sphere and the first input port of the second big integrating sphere;
a first complementary color adjusting system comprising a light filtering disc and a light blocking disc and disposed between the first exit port of the second small integrating sphere and the second input port of the first big integrating sphere;
a second complementary color adjusting system comprising a light filtering disc and a light blocking disc and disposed between the second exit port of the second small integrating sphere and the second input port of the second big integrating sphere;
a beam splitting system comprising of a cubic splitting prism having a first and a second adjacent planes, a target plate, a first reticle and a second reticle;
a means for measuring a color difference threshold;
a means for measuring an illumination threshold; and
a means for measuring a contrast threshold.
